(19) United States
(12) Patent Application Publication
(10) Pub. No.: US 2023/0002810 A1
(43) Pub. Date: Jan. 5, 2023

(54) Method for Carrying Out an Amplification Reaction in a Microfluidic Apparatus

(71) Applicant: Robert Bosch GmbH, Stuttgart (DE)

(72) Inventors: Franz Laermer, Weil Der Stadt (DE); Martin Schulz, Winterbach (DE)

(21) Appl. No.: 17/757,132

(22) PCT Filed: Dec. 10, 2020
(86) PCT No.: PCT/EP2020/085602

§ 371 Date: Jun. 9, 2022

(30) Foreign Application Priority Data
Dec. 13, 2019 (DE) 10 2019 219 531.8

(51) Int. Cl. C12Q 1/6818; C12Q 1/6848; C07D 237/30

(52) U.S. Cl. CPC C12Q 1/6818; C12Q 1/6848; C07D 237/30; B01L 3/502715

(57) ABSTRACT

In an amplification reaction in a microfluidic apparatus, the reaction is carried out using starting substances tagged with fluorophore and quencher. The detection of reaction products occurs according to the disclosure by a separation of fluorophore and quencher occurring in the context of the amplification reaction. For the detection reaction, at least one energy-transferring substance is added and the evaluation occurs on the basis of the fluorescence emission of the fluorophores which occurs.

The present invention relates to a method for carrying out an amplification reaction in a microfluidic device, wherein the reaction is carried out using starting substances labeled with a fluorophore and a quencher and a chemical energy source for excitation of the fluorophores by means of energy transfer. The invention further relates to a kit and to a microfluidic device for carrying out such an amplification reaction.

### PRIOR ART

The polymerase chain reaction (PCR) is a quick and highly sensitive method in DNA analysis which was developed in the 1980s. Here, specific primers are used to carry out a defined enzymatic in vitro replication of the DNA, in which the target sequence is multiplied (amplified) in multiple cycles. Following on from the radioactive labeling of the reaction products which was originally used, the use of fluorescence to detect the reaction products has now gained acceptance here. For this purpose, the reaction can be carried out using starting substances labeled with a fluorophore and a quencher. The fluorescence is generated by optically exciting the fluorophores or fluorescent dyes with an appropriate excitation wavelength. When a quencher is located in the spatial proximity of the fluorophore, the fluorescence is deactivated non-radiatively. When the fluorophore and quencher are spatially separated from one another in the course of the amplification reaction taking place, the fluorescence is measurable.

For the reaction it is possible to use, for example, primer chains or what are known as TaqMan probes which are labeled with a fluorophore and which contain a fluorophore and a quencher in spatial proximity. These chains or probes are incorporated and/or hydrolyzed during the polymerase chain reaction, with the result that the spatial proximity between the fluorophore and quencher no longer occurs and it is possible to detect a fluorescence from the reaction products after optical excitation. The excitation light and the fluorescence emission generally have different wavelengths (what is known as the “Stokes shift” between absorption and emission maxima). These wavelengths can be separated from one another using optical filters in a detection device, with the result that a detector or a detection camera ideally detects only the light having the wavelength of the fluorescence emission.

### DISCLOSURE OF THE INVENTION

### Advantages of the Invention

The proposed method serves for carrying out an amplification reaction in a microfluidic device, for example on a lab-on-a-chip within what are known as silicon microarray cells. Such microfluidic devices are already used, for example, in medical diagnostics. The amplification reaction is carried out using starting substances labeled with a fluorophore and a quencher. The fluorophore and quencher separate in the course of the amplification reaction, as a result of which it is possible to detect reaction products or to evaluate the amplification that has taken place by way of the detectable fluorescence emission. According to the proposed method, the detection is performed on the basis of the fluorescence emission by adding at least one energy-transferring substance and evaluating the fluorescence emission of the fluorophores that occurs. Unlike in conventional methods, in the proposed method it is not necessary to optically excite the sample volume so as to enable a read-out of the fluorescence. Rather, the fluorophores are excited by transfer of chemical energy with the aid of the energy-transferring substance.

The energy-transferring substance is preferably one or more luminescent substances. The basis of the luminescence is that corresponding luminescent substances may be optically, electrically or chemically shifted into an excited energy state. Said excited energy state of the luminescent substances is generally long-lasting, i.e. these are generally energy states from which an optical transition to a ground state is prohibited, for example due to dipole selection rules. The excited energy state may for example be what is known as a triplet state (T1), from which an optical transition to a singlet ground state (S0) is prohibited. The present invention utilizes the relatively long-lasting chemical excitability of luminescent substances, since the energy from the long-lasting excited states can be transferred to other molecules particularly easily, in order for example to excite said fluorophores to emit fluorescence by way of energy transfer. The luminescent substances themselves are generally excited by way of a chemical reaction, for example an oxidation reaction with or without a catalyst. It is thus the central point of the proposed method that the fluorescence is not excited optically, rather that energy is transferred to the fluorophores with the aid of energy-transferring substances, and so the fluorescence emission of the fluorophores is evaluable in the absence of quenchers in the direct vicinity. The excitation energy is thus supplied to the quenched or non-quenched fluorophores after the amplification reaction via a chemical energy source, in particular via a luminescent substance. Other chemically excitable substances are also suitable in principle as energy transferers for the purposes of the invention. It is a prerequisite here that the excited state is relatively long-lasting, so as to be able to transfer the energy to the fluorophores reliably and with good efficiency. Furthermore, the substances acting as energy transferers should expediently be water-soluble so that they are usable in buffer systems, as are used for such reactions.

This novel combination of a chemical energy-carrying molecule with suitable fluorophores makes it possible to dispense with optical excitation. Optical excitation for generation of the fluorescence is often problematic or complicated in particular in the case of microfluidic devices since as large as possible an area of the microfluidic device must be irradiated with light of a defined intensity and wavelength as homogenously as possible. This requires complicated and expensive optics which are not desired for cost reasons in the case of microfluidic devices, in particular with regard to point-of-care devices and/or devices with a high throughput. The use of energy-transferring substances according to the proposed method for excitation of the fluorophores makes it possible to dispense with such complicated and expensive optics when carrying out the amplification reactions. The proposed method thus allows a method for optical read-out for an amplification reaction, for example for a PCR reaction and/or an isothermal amplification reaction, in microfluidic devices without the need for an optical light source and corresponding optical devices for excitation of a light emission.

The luminescent substance may for example be 3-aminophthalhydrazide (5-amino-2,3-dihydrophthalazine-1,4-dione; known as luminol) and/or 3-nitrophthalhydrazide. Such luminescent substances are distinguished in that they are shifted into an excited state by way of supplied energy particularly via an oxidation reaction and thereby emit light. Since the excited energy states are long-lasting, the luminescence of said substances is comparatively weak. The wavelength corresponding to the light emission (luminescence radiation) or the corresponding amount of energy is utilized in the context of the proposed method so as to be transferred non-radiatively to the fluorophores and thus to excite the fluorescence of the fluorophores that have separated from the quenchers. In principle, the energy is transferred here regardless of the luminescence radiation.

The reaction that leads to the molecular excitation and subsequently to the energy transfer is preferably based on a chemical reaction, in particular on an oxidation reaction. In this respect, the energy-transferring substance acquires its chemical excitation energy particularly from a (catalyzed) oxidation reaction. The oxidant used may in particular be hydrogen peroxide or carbamide peroxide (=hydrogen peroxide-urea), and so the detection in the context of the proposed method is preferably carried out in the presence of hydrogen peroxide. The particularly preferred energy-transferring substance luminol is transferred, as chemical energy source, into a long-lasting excited (triplet) state by way of an oxidation with hydrogen peroxide. This stored excitation energy can then be transferred to other fluorophores, in particular those emitting at longer wavelengths, with the result that the fluorescence is made possible as proof of the amplification reaction. This transfer of energy from the only weakly luminescent luminol or related molecules to a highly fluorescent molecule, such as a Rhodamine or Yakima Yellow, leads overall to an increase in the light emission, which is why this is also referred to as “chemical sensitization”.

The oxidation reaction that leads to the excitation of the luminescent substances is driven by the decomposition of hydrogen peroxide. The hydrogen peroxide may be present, for example, in the form of carbamide peroxide, i.e. as a mixture of hydrogen peroxide and urea. Furthermore, in addition or as an alternative, alkaline conditions may be established for the detection, with for example at least one alkaline substance being contained in the reaction liquid that contains the luminescent substance. By way of example, dilute sodium hydroxide solution or sodium hydrogencarbonate solution, which increases the solubility of the luminescent substances, such as luminol, and simultaneously accelerates the decomposition of hydrogen peroxide, may be contemplated. Use may alternatively be made of corresponding buffers.

The detection is particularly advantageously performed in the presence of at least one catalyst that catalyzes the chemical reaction on which the energy-transferring process is based. The catalyst thus preferably catalyzes an oxidation reaction. The catalyst may preferably be potassium hexacyanoferrate(III) and/or manganese peroxide and/or hydroquinone or related substances, for example catechol or resorcinol. Since some catalysts do not interact with the actual amplification reaction, it may be contemplated in particularly preferred embodiments that such a PCR-compatible (or amplification-compatible) catalyst is already initially introduced into the microfluidic device of the microarray cells. Hydroquinone is particularly suitable in this context, for example. In other configurations, use may for example be made of enzymatic catalysts, e.g. horseradish peroxidase (HRP).

Preferably, the at least one energy-transferring substance is added by overcoating the microfluidic device (for example the silicon microarray chip) with a reaction liquid in which the at least one energy-transferring substance and optionally further components, such as hydrogen peroxide and optionally a catalyst, are contained. “Overcoating the microfluidic device” here means that the reaction liquid is introduced into the microfluidic device particularly after completion of the amplification reaction, so that overcoating takes place above the reaction spaces of the microfluidic device. “Reaction liquid” here means the liquid in which the excitation of the energy-transferring substances is performed, i.e. in particular the excitation of the luminescent substances. What is preferably concerned here is an oxidation reaction for excitation of the luminescent substances, which can subsequently transfer their chemical energy to the fluorophores. Overcoating has the particular advantage that the oxidation reaction and the transfer of energy to the fluorophores take place in principle only at the interface of the liquids where the different components mix by diffusion, with the result that the reaction used for the evaluation can be more efficiently utilized timewise and the fluorescence generated can be detected over a longer period.

This configuration of the proposed method is thus concerned with the use of a chemically excited luminescence reaction for excitation of the fluorescence that is used as a read-out or detection reaction for an amplification reaction. This combination is very advantageous with regard to microfluidic devices since the optical excitation that can often be realized only with difficulty for microfluidic devices can be dispensed with and replaced by addition of the corresponding reagents for the luminescence reaction. This can be carried out in a simple manner by overcoating with a corresponding reaction liquid after completion of the amplification reaction in the sense of an endpoint reaction. Such overcoating is suitable in a simple manner for automation, and therefore this method is suitable particular advantages with regard to the use of microfluidic devices, for example in medical diagnostics, with a high throughput and with little equipment complexity.

Suitable fluorophores are generally fluorescent dyes that emit in a range with a longer wavelength than the luminescent substances preferably used. This has the advantage that the interfering luminescence radiation and the fluorescence radiation that is of interest for the actual evaluation can easily be separated from one another by optical filters, so that the interfering luminescence background radiation can in a way be masked for the evaluation. Particularly suitable fluorophores are Rhodamine B and/or other Rhodamines and/or Yakima Yellow and/or Cy5, with particular preference being given to Yakima Yellow. The emission maximum of Yakima Yellow is 550 nm and extends beyond 600 nm. The absorption maximum of Yakima Yellow is 525 nm. The maximum of the luminescence emission of luminol is 425 nm and longer wavelengths, and therefore non-radiative energy transfer to Yakima Yellow can easily take place. The wavelength of the fluorescence emission of Yakima Yellow (550 nm or more) and the wavelength of the “interfering” luminescence emission of luminol (425 nm or more) differ to such a significant extent, however, that they can easily be separated from one another by an optical filter, so that the luminescence emission of luminol can be masked for the evaluation.

In particularly preferred configurations of the method, the evaluation is performed using at least one optical filter for removing interfering luminescence radiation from the fluorescence radiation. The optical filter is expediently adapted to the particular substances used and the emission wavelengths thereof, i.e. in particular the luminescence emission wavelength and the fluorescence emission wavelength, so that, in the case of luminescent substances, the luminescence emission wavelength is filtered out. For the filtering, use may for example be made of an optical bandpass filter having a transmission wavelength of for example 570 nm and/or an optical edge filter having a transmission wavelength from for example 570 nm or longer wavelengths, so that, when using luminol and Yakima Yellow, the two emissions of this example can be separated from one another with a particularly high level of efficiency.

In a particularly preferred configuration, the amplification reaction is an endpoint reaction, i.e. in particular an endpoint PCR or an endpoint reaction of an isothermal amplification reaction. When carrying out the method “to the endpoint”, no read-out is performed during the course of the amplification reaction, rather it is not until the end of the amplification reaction that the detection reaction is started by adding the corresponding substances or overcoating with the corresponding substances, in particular the luminescent substances and the substances required to initiate the luminescence reaction. After completion of the amplification reaction, i.e. when the multiplication reaction in the reaction spaces of the microfluidic device has reached its endpoint, the microfluidic device may be overcoated with the reaction liquid containing the energy-transferring substances and optionally further substances, such as a catalyst and hydrogen peroxide as oxidant. It is also possible and advantageous to already have the catalyst available in the PCR chemistry. For the latter case it is necessary to select PCR-compatible catalysts, i.e. substances that do not adversely affect the PCR. For example, hydroquinone is suitable for this purpose. The energy-transferring substance is generated as a result, and so the chemical energy thereof is transferred to the fluorophores. Provided that the fluorophores are spatially separated from their quenchers as a result of the amplification reaction that has taken place, the fluorescence is detectable as proof of the amplification reaction that has taken place. Overall, the light emission of the fluorescence is therefore proportional to the amount of quencher-free fluorophores, which in turn is proportional to the amplification reaction that has taken place. The fluorescence light emission is thus proportional to the amount of products formed, and therefore the fluorescence makes it possible to evaluate the amplification reaction.

The invention further encompasses a kit for carrying out an amplification reaction in a microfluidic device. Said kit preferably comprises two reaction mixtures, the first reaction mixture containing components for carrying out the amplification reaction and the second reaction mixture containing components for carrying out the detection reaction.

The first reaction mixture especially contains the starting substances that are suitable for a fluorescence read-out of the reaction and that are in particular labeled with one or more fluorophores and quenchers, for example correspondingly labeled primers or probes. Furthermore, the first reaction mixture may contain customary substances for carrying out an amplification reaction, for example for a customary PCR reaction or an isothermal amplification reaction, e.g. buffers, nucleotides, primers and/or DNA polymerase. These customary substances for carrying out the amplification reaction may optionally be included or, depending on the application, be provided and added by the user. Furthermore, the first reaction mixture may also contain a PCR-compatible catalyst for the subsequent oxidation reaction (detection reaction). To carry out the reaction, it may also be contemplated that the user adds the PCR-compatible catalyst to a standard PCR kit for the subsequent oxidation reaction. Furthermore, the first reaction mixture may already contain at least one energy-transferring substance, by means of which the detection reaction according to the proposed method is carried out. The energy-transferring substance used is preferably a luminescent substance, in particular luminol. The oxidant, such as hydrogen peroxide or carbamide peroxide, is provided separately or in the second reaction mixture of the kit, since it would interfere with the amplification reaction.

The second reaction mixture of the kit provides components which preferably also contain the substances that ensure the reaction with which the energy-transferring substances are excited. These are in particular the substances for an oxidation reaction, for example hydrogen peroxide or carbamide peroxide as oxidant and optionally one or more catalysts for the oxidation reaction and/or suitable buffer substances, for example to establish suitable alkaline conditions. As already mentioned, the catalyst, provided it is PCR-compatible, may also already be a component of the above-described first reaction mixture of the kit. The amplification reaction can be carried out and evaluated in the manner described above using the reaction mixtures of the kit, with the energy-transferring substance, and in particular the luminescent substance, being used to transfer chemical energy to the fluorophores which can then emit light (fluorescence) provided that no quencher is nearby. With regard to further features of the kit for carrying out the amplification reaction, reference is made to the above description.

Finally, the invention encompasses a microfluidic device for carrying out amplification reactions. Said device is characterized in that it is configured to carry out the proposed method. Said device may in particular be a lab-on-a-chip containing a silicon chip with a multiplicity of microarray cells, as are already known for carrying out miniaturized reactions, for example molecular diagnostic reactions in medical diagnostics. Said microfluidic device is configured to carry out an amplification reaction with a fluorescence read-out, with starting substances labeled with one or more fluorophores and a quencher being used in the context of the reaction. This involves the excitation of the fluorescence not, as in conventional methods, by way of optical excitation, but rather by way of an energy-transferring reaction from chemically excited molecules, in particular luminescent molecules, with the result that chemical energy is transferred to the fluorophores, leading to emission of the fluorescence radiation provided that there is no quencher in the vicinity of the fluorophore. The microfluidic device configured to carry out this combined reaction is distinguished in particular in that, for example, a catalyst and/or an oxidant required for the reaction for excitation of the energy-transferring substances is/are initially introduced. Corresponding buffers may also be initially introduced. The energy-transferring substances are generally only added after completion of the amplification reaction in the sense of an endpoint reaction, for example by overcoating with a corresponding reaction liquid containing the corresponding substances. With regard to further features of said microfluidic device, reference is made to the above description.

Further features and advantages of the invention are revealed by the following description of exemplary embodiments in conjunction with the drawings. The individual features can each be realized here on their own or in combination with one another.

In the drawings:

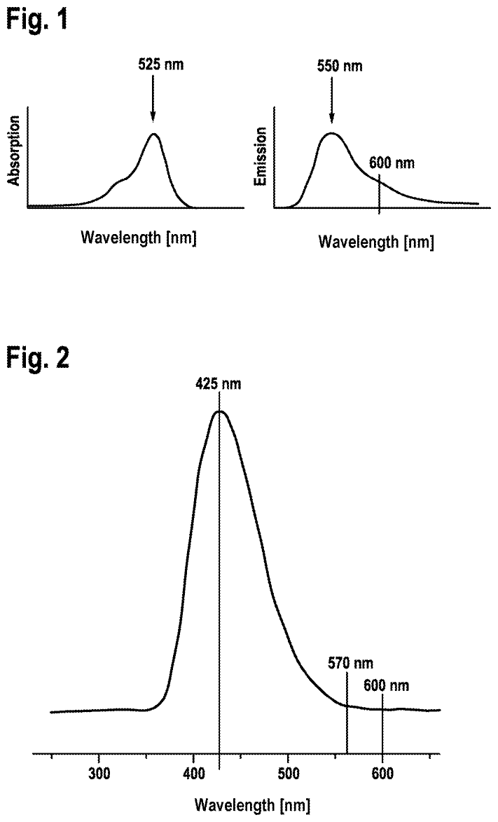

FIG. 1 shows spectra for the absorption and emission of the Yakima Yellow fluorophore, and

FIG. 2 shows an emission spectrum of luminol.

### DESCRIPTION OF EXEMPLARY EMBODIMENTS

The proposed method provides a new method for carrying out an amplification reaction in a microfluidic device, in which method the detection method known per se for the amplification reaction based on fluorescent dyes is combined with chemical excitation, in particular non-radiative excitation of the fluorophores by transfer of energy from excited molecules. In the case of the fluorescence method that is known per se and used in connection with amplification reactions, starting substances labeled with a fluorophore, for example fluorophore-labeled primer chains or TaqMan probes, are incorporated or hybridized or hydrolyzed during the amplification reaction. These primers or probes contain one or more fluorophores and a quencher which are directly adjacent. For as long as this configuration of fluorophore and directly adjacent quencher exists, no fluorescence can be emitted. In particular, no fluorescence can be emitted from such primer chains or probes, in each case bearing a fluorophore and quencher, for as long as they are located freely in the solution. It is only the incorporation of such primer chains or probes into an amplification product that causes either the splitting and spatial separation of fluorophore and quencher or the cleavage of the quencher from the molecule bearing the fluorophore. Such fluorophores can then emit fluorescence radiation after excitation and represent a measure of the amount of amplification products formed. This method makes it possible, by way of the transfer of energy to the fluorophores on the basis of a chemical reaction, to dispense with complicated and expensive optics that were conventionally required for optical excitation in the context of the fluorescence detection reaction.

The proposed method can be carried out in particular for an endpoint PCR reaction or an isothermal endpoint amplification reaction in singleplex or multiplex in a silicon microarray or in some other microfluidic device, wherein the amplification reaction involves the described incorporation of molecule pairs consisting of quenchers and fluorophores and/or the hydrolysis of such starting substances during the formation of the amplification products. After completion of the amplification reaction, that is to say as soon as the amplification reaction in the array cells has essentially reached its endpoint, the microfluidic device can be overcoated with a reaction liquid containing the energy-transferring substances, in particular the luminescent substances. After they have been excited, said substances can carry chemical energy for a relatively long period of time (regardless of their luminescence), and so this energy can be transferred non-radiatively to the fluorophores.

The reaction on which the excitation of the energy-transferring substances is based, in particular the oxidation reaction, can be accelerated by a catalyst. Particularly suitable for this purpose are red prussiate (potassium hexacyanoferrate(III)) or hydroquinones or other oxidation catalysts. Said catalyst may be admixed with the reaction mixture consisting of hydrogen peroxide, luminol and suitable buffers. Some catalysts may also be prestored directly in the array cells of the microfluidic device provided that they do not interfere with the actual amplification reaction, this being the case for example for many hydroquinones. Other options for suitable catalysts include, for example, manganese peroxide (manganese dioxide) that may also be prestored in the array cells particularly in the form of a suspension. Manganese peroxide is not water-soluble and therefore does not interfere with the amplification reaction. On contact with hydrogen peroxide, manganese peroxide catalyzes the decomposition of the hydrogen peroxide molecules and the oxidation of the energy-transferring substances, for example of luminol, to give the long-lasting excited form. Furthermore, manganese peroxide is a very cost-effective and safe compound that is therefore particularly suitable as a solid catalyst for the decomposition of hydrogen peroxide and the initiation of the subsequent oxidation of luminol.

Suitable fluorophores are in principle all fluorescent dyes that emit with a greater wavelength than the energy-transferring or luminescent substance used. By way of example, suitable fluorophores that may be used in combination with luminol as luminescent substance are Rhodamine B, other Rhodamines, Yakima Yellow or Cy5. FIG. 1 illustrates absorption and emission spectra of Yakima Yellow. Here the absorption maximum is 525 nm and the emission maximum is 550 nm and the emission extends to significantly longer wavelengths beyond 600 nm. FIG. 2 depicts the emission spectrum of luminol: the emission maximum is 425 nm and the emission extends to significantly greater wavelengths. The excitation energy of luminol can thus lead to excitation of Yakima Yellow. It is advantageous for the evaluation of the fluorescence emission of Yakima Yellow that the two emissions, i.e. the interfering luminescence emission of luminol and the fluorescence emission of Yakima Yellow, can easily be separated from one another by optical filters, so that the luminescence emission of luminol does not interfere with or overlay the fluorescence emission that is relevant for the evaluation of the detection reaction.

Customary probes with the Yakima Yellow fluorophore and a quencher are based in principle on a bridge structure that couples the quencher to the fluorophore. When a probe that has been labeled in this way is annealed or attached to a DNA strand, the bridge opens, as a result of which the quencher is spatially separated from the Yakima Yellow fluorophore.

The type of read-out in the sense of an endpoint PCR or an isothermal endpoint amplification reaction can be technically implemented without any great effort. Although the individual reaction does not allow any quantitative statement to be made, the “quantitative” feature is generally no longer necessary with the high degrees of multiplexing that are possible. The particular advantage of the proposed method, where optical excitation of the sample volumes is dispensed with, makes it possible for example to process and read even large-area silicon microarrays having a very large number of array cells and different detection reactions in parallel.

When carrying out the method, an amplification reaction that is known per se with primers and/or probes that are suitable for a fluorescence read-out can be carried out first. After the endpoint of the amplification reaction has been reached, the reaction mixture for the detection reaction according to the invention, for example consisting of water, luminol, hydrogen peroxide/urea (carbamide peroxide) and red prussiate (potassium hexacyanoferrate(III)) as catalyst for the reaction leading to the excitation of the energy-transferring substances, in the microfluidic device can be slowly fed to the PCR multiarray chip, so as to thereby slowly overcoat the reaction mixture of the amplification reaction. To assist the detection reaction, in particular to destabilize the hydrogen peroxide and to increase the solubility of luminol, it is for example also possible to add a small amount of sodium hydroxide solution or alkaline buffer (e.g. sodium hydrogencarbonate), it also being possible to contemplate prestorage of suitable buffers in the microfluidic device.

The substances for the reaction that serves for the excitation of the energy-transferring substances may be directly added at the start of the detection reaction or in some cases already prestored in the array cells or elsewhere in the lab-on-chip system. It is a particular advantage of such lap-on-chip systems that all the reagents required can be prestored therein at a suitable location and in a suitable manner. In particular, a PCR-compatible catalyst such as hydroquinone is suitable for prestorage in the array cells of the silicon chip as hydroquinone does not interfere with the amplification reaction. Another particularly suitable option is the prestorage of manganese peroxide (manganese dioxide) as water-insoluble solid catalyst in the array cells. Manganese peroxide, as a water-insoluble substance, also generally does not interfere with the amplification reaction that takes place in aqueous solution. Because manganese peroxide is a very efficient decomposition catalyst for hydrogen peroxide and hence for the oxidation of the luminol, manganese peroxide is also particularly suitable as a starting point for the chemical excitation of the luminescent substance, in particular of luminol. The excited luminol transfers its energy to the fluorophore, for example Yakima Yellow. Provided that the Yakima Yellow fluorophore is spatially separated from its quencher, that is to say in particular when the Yakima Yellow primer or the Yakima Yellow probe is bound to a DNA strand and the quencher is thereby removed, Yakima Yellow can fluoresce. For the read-out of the fluorescence radiation, use is preferably made of an optical filter (e.g. 570-580 nm), optionally combined with an additional edge filter that blocks emission with a wavelength shorter than 580 nm, in order to suppress the luminescence radiation of luminol that interferes with the evaluation.

Reaction mixtures used to carry out the PCR in the array cells are, for example, commercially available proprietary PCR kits from manufacturers which are themselves not subject matter of the present invention. The PCR kit selected is optionally supplemented by suitable PCR-compatible catalysts and additional buffer substances in order to optimally adjust said kit to the environment of the array cells in the silicon chip. Furthermore, the PCR kit should preferably work with Yakima Yellow or a suitable Rhodamine as the detection fluorophore in the sense of the present description.

An exemplary reaction mixture for the overcoating of the PCR kit after completion of the PCR in the array cells to trigger the detection reaction may comprise the following components and concentrations:
Ultrapure water
Hydrogen peroxide, 0.1%-10% (corresponding to 30 mmolar-3 molar), preferably 0.5%-5% (corresponding to 150 mmolar-1.5 molar)
Alternatively carbamide peroxide (hydrogen peroxide-urea), 30 mmolar-3 molar, preferably 150 mmolar-1.5 molar
Sodium hydroxide, 10 mmolar-1 molar, preferably 50-500 mmolar, particularly preferably 100-150 mmolar
Alternatively sodium hydrogencarbonate, 50 mmolar-1 molar, preferably 100-500 mmolar
Luminol, 10 mmolar 150 mmolar, particularly preferably 50-100 mmolar
Catalyst (hydroquinone, horseradish peroxidase HRP, manganese peroxide, potassium hexacyanoferrate(III)), 10 mmolar-1 molar, preferably 50 mmolar-500 mmolar, particularly preferably 100 mmolar

1. A method for carrying out an amplification reaction in a microfluidic device, comprising:
carrying out the amplification reaction using starting substances labeled with a fluorophore and a quencher; and
detecting reaction products resulting from a separation of the fluorophore and quencher that has taken place in the course of the amplification reaction by adding at least one energy-transferring substance and evaluating the fluorescence emission of the fluorophores that occurs.

2. The method as claimed in claim 1, wherein the energy-transferring substance is a luminescent substance.

3. The method as claimed in claim 2, wherein the luminescent substance is 3-aminophthalhydrazide and/or 3-nitrophthalhydrazide.

4. The method as claimed in claim 1, wherein the detection is carried out in the presence of hydrogen peroxide.

5. The method as claimed in claim 4, wherein the hydrogen peroxide is used in the form of carbamide peroxide.

6. The method as claimed in claim 1, wherein the detection is performed in the presence of at least one catalyst.

7. The method as claimed in claim 6, wherein the catalyst is potassium hexacyanoferrate(III) and/or manganese peroxide and/or hydroquinone and/or catechol and/or resorcinol and/or horseradish peroxidase (HRP).

8. The method as claimed in claim 6, wherein the catalyst is initially introduced into the microfluidic device.

9. The method as claimed in claim 1, wherein the addition of the at least one energy-transferring substance includes adding the at least one energy-transferring substance by overcoating with a reaction liquid in which the at least one energy-transferring substance is contained.

10. The method as claimed in claim 1, wherein the fluorophore used is Rhodamine B, another Rhodamine, and/or Yakima Yellow and/or Cy5.

11. The method as claimed in claim 1, wherein the evaluation is performed includes using at least one optical filter.

12. The method as claimed in claim 1, wherein the amplification reaction is an endpoint reaction.

13. A kit for carrying out an amplification reaction in a microfluidic device, comprising:
starting substances labeled with a fluorophore and a quencher; and
at least one energy-transferring substance configured for a detection reaction.

14. The kit as claimed in claim 13, wherein:
the starting substances labeled with a fluorophore and a quencher are configured for carrying out the amplification reaction; and
the at least one energy-transferring substance is configured to detect reaction products resulting from a separation of the fluorophore and quencher that has taken place in the course of the amplification reaction and to enable evaluating the fluorescence emission of the fluorophores that occurs.

15. A microfluidic device for carrying out amplification reactions, comprising:
starting substances labeled with a fluorophore and a quencher and configured to carry out the amplification reaction; and
at least one energy transferring substance configured to detect reaction products resulting from a separation of the fluorophore and quencher that has taken place in the course of the amplification reaction and to enable evaluating the fluorescence emission of the fluorophores that occur.
